(19) United States
(12) Patent Application Publication
(10) Pub. No.: US 2003/0113304 A1
(43) Pub. Date: Jun. 19, 2003

(54) Selective tissue site revascularization by combined focal injury and hematopoietic stem cell introduction

(75) Inventor: Daniel Burkhoff, Tenafly, NJ (US)

Correspondence Address: John P. White, Cooper & Dunham LLP, 1185 Avenue of the Americas, New York, NY, 10036, US

(73) Assignee: The Trustees of Columbia University

(21) Appl. No.: 10/299,079

(22) Filed: Nov. 19, 2002

Related U.S. Application Data
(63) Continuation of application No. 09/090,775, filed on Jun. 4, 1998, now abandoned.
(63) Continuation-in-part of application No. 08/483,512, filed on Jun. 7, 1995, now Pat. No. 5,840,059.

(51) Int. Cl. A61K 48/00; C12N 5/08

(52) U.S. Cl. 424/93.21; 435/372

(57) ABSTRACT

The effects of angiogenesis created by tissue injury resulting from ischemic tissue being revascularized by transmyocardial revascularization are amplified by introducing cells containing an angiogenic agent into a patient positive to antigenic determinant such as Fik-1, Tie-2 or CD-34. Cells such as CD 34+ cells migrate to a site of angiogenesis and will thus efficiently carry the angiogenic agent to the site without requiring local introduction. Thus, a section of ischemic myocardium can be revascularized by performing TMR to create a number of sites of injury, and then introducing hematopoietic stem cells, for example CD 34+ cells, that contain a growth factor, genetic material or other angiogenic agent into the patient, most preferably by systemic introduction. As a result, the increase in vascularization will be greater than that exhibited by TMR alone or by the insertion of an angiogenic agent alone.

### RELATED APPLICATIONS

This application is a continuation-in-part of U.S. patent application 483,512, filed Jun. 7, 1995 and entitled “Therapeutic and Diagnostic Agent Delivery” which is incorporated by reference herein as if set forth in its entirety.

The present invention relates to revascularization of specific tissue sites via vasculogenesis and/or angiogenesis, and more specifically relates to myocardial revascularization.

### BACKGROUND OF THE INVENTION

The study of the biological mechanisms of vessel growth is important both as an aid to understanding how to inhibit such growth, as is the case with inhibiting the vascularization of tumors, and as to how vascularization may be encouraged. In the latter case, new vessels may be needed to nourish implanted tissue or regenerated tissue, or to revitalize ischemic tissue. A prime example of such an indication is ischemic myocardium in patients suffering from advanced coronary artery disease (CAD) in which the native vessels have become occluded by atheromatous plaque that prevents perfusion of the myocardium distal to the occlusions. CAD is a major health problem affecting millions of persons worldwide.

There are a number of medical treatments to limit, remove or mitigate arterial occlusions and alleviate the symptoms of CAD. One treatment modality is medical therapy in which medication is used to limit the progression of the occlusive plaque, relieve the symptoms associated with the occlusion or, in some cases, reverse the occlusion by diminishing the plaque. There are also a number of invasive techniques that do not rely solely on medication such as coronary artery bypass grafting (CABG), percutaneous transluminal balloon angioplasty (PTCA) and transmyocardial revascularization (TMR). In one type of TMR treatment, a laser is used to create a number of channels in the myocardium that are approximately one millimeter in diameter. It is not entirely certain whether these channels remain patent and perfuse the ischemic myocardium, however, the creation of the channels provides relief from angina pectoris, and the channels become sites of revascularization. More broadly, TMR treatment in other forms involves creating a number of sites of focal injury in ischemic tissue using laser energy or other sources of energy.

Although TMR is an effective therapy, further enhancement of these benefits may be obtained by combining TMR with an angiogenic agent, such as a vascular endothelial growth factor (VEGF) or basic fibroblast growth factor (bFGF), as well as genetic constructs and other known angiogenic substances in order to enhance the effects of revascularization. Such combined therapies are disclosed in U.S. patent application 483,512, filed Jun. 7, 1995 and entitled “Therapeutic and Diagnostic Agent Delivery” which is assigned to the assignee of the present invention and is incorporated herein by reference. Previously, however, the delivery of angiogenic agents has been focused on. localized delivery systems that ensure that the angiogenic agents reach the channels or sites of focal injury created by the TMR procedure. Systemic introduction was not favored since angiogenic agents are likely to induce undesirable physiological effects, e.g., acute hypotension or retinopathy.

The isolation of putative progenitor endothelial cells for angiogenesis is known. T. Asahara, et al., Science 1997 Feb. 14;275(5302):964-967. The progenitors discussed were isolated on the basis of cell surface antigen expression and were found, in ischemic animal models, to have been incorporated into sites of active angiogenesis. Because the progenitors home to foci of angiogenesis they have been suggested to be useful as autologous vectors for gene therapy. For example, in the case of unilateral hindlimb ischemia, angiogenesis could be amplified by transfection of CD 34 positive mononuclear blood cells to achieve constructive expression of angiogenic cytokines or provisional matrix proteins or both.

Thus, it is known that the identified endothelial cell progenitors are useful to deliver angiogenic agents. There remains, however, a long-felt and as of yet unsolved need to provide methods and apparatus whereby a patient may be actively treated to enhance the delivery of heterologous genetic constructs that proliferate vessel growth to sites where it is specifically desired to generate new vessel growth. Moreover, it would be desirable to provide a system and therapy whereby a patient would be treated to revascularize ischemic myocardium in a manner that amplifes the effects of TMR without requiring localized delivery of a substance into the myocardium itself.

### SUMMARY OF THE INVENTION

It has now been found that specific tissue sites can be vascularized by selecting cells with an affinity to sites of angiogenesis and introducing such cells as part of TMR therapy. The present invention provides a therapy that comprises creating one or more sites of focal injury; and delivering hematopoietic stem cells to the patient to enhance vascularization. In a preferred embodiment, CD34+ cells that have been engineered to contain an angiogenic agent are introduced into the patient, most preferably by systemic injection.

### DETAILED DESCRIPTION OF THE PREFERRED EMBODIMENTS

“Vascularizing” as used herein is meant in its broadest sense and includes creating vessels in ischemic and non-ischemic tissue, either via the mechanism of angiogenesis or vasculogenesis. In a particularly preferred embodiment, ischemic myocardium is targeted and revascularized as part of either percutaneous or intraoperative transmyocardial revascularization therapy. As known in the art, transmyocardial revascularization can be performed as part of an intraoperative procedure, e.g., using a thoracotomy, or as part of a minimally invasive procedure. In either approach, the sites of focal injury can be non-transmural or, preferably, are transmural. Alternatively, a percutaneous technique may be employed wherein non-transmural sites of focal injury are created in the endocardium and preferably into the myocardium. Regardless of the technique employed to create the focal injury or injuries, a number of energy modalities are known to be useful, and all are considered part of the present invention. A presently preferred technique is to deliver laser energy to the heart tissue in order to ablate tissue, forming a channel. However, the amount of tissue disturbed or ablated may be altered and it is neither necessary to ablate tissue nor to create a patent lumen or lacuna that could be denoted a “channel” since tissue injury is a mechanism involved in the beneficial effects of TMR. In addition to using laser energy, other forms of electromagnetic energy such as radiofrequency ablation, fulguration or other forms of direct current are useful, as are ultrasonic ablation and even mechanical dissection. In accordance with the present invention, all that is required is the creation of an injury at a site where increased vascularity is desirable.

As used herein, the term “angiogenic agent” includes any substance useful in a procedure that promotes the growth of new vessels, particularly the growth of new vessels in the myocardium, however, it will be understood that an angiogenic effect is useful in other organs, such as the liver and kidneys. The present invention may employ a wide variety of angiogenic agents, including small molecule drugs, active compounds, gene products and genetic therapy agents, as well as cytokines or provisional matrix proteins or both. Examples of active compounds include, by way of non-limiting example, biologically active carbohydrates, recombinant biopharmaceuticals, agents that are active in the regulation of vascular physiology, such as nitric oxide agents that effect the regulation of gene activity by modulating transcription, the turnover of cellular mRNA, or the efficiency with which specific mRNA is translated into its protein product, i.e., antisense pharmaceuticals. Other active compounds include hormones, soluble receptors, receptor ligands, peptides (both synthetic and naturally occurring), peptidomimetic compounds, specific and non-specific protease inhibitors, postaglandins, inhibitors of prostaglandin synthase and/or other enzymes involved in the regulation of prostaglandin synthesis, growth factors that affect the vascualture such as acidic and basic fibroblast growth factors (bFGF), FGF, vascular endothelial growth factor (VEGF), andgiogenin, transforming growth factor alpha, and transforming growth factor beta. The foregoing list is meant to illustrate the breadth of angiogenic agents and other substances useful with the present invention and is not meant to be exhaustive or in any way limit the scope of the claims herein. It is contemplated that there are classes of angiogenic agents possessing structures significantly similar to other molecular agents, and that these agents will have specific biological activities associated with them while being deficient in other biological activities that are less desirable therapeutically. Any and all of the angiogenic agents useful with the present invention may comprise substantially pure compounds, defined or relatively less well defined admixtures of compounds, such as those that might result from a biological system such as conditioned serum or conditioned cell culture media.

Upon creation of one or more sites of focal injury, certain types of hematopoietic stem cells that have been delivered to the patient will migrate and concentrate at the sites of focal injury. Native cells that are CD34+ cells will migrate to sites of angiogenesis, and therefore CD34+ cells are particularly useful in this regard, as they display a high degree of affinity for sites at which a myocardial injury such as a laser channel has been created. Similarly, other antigenic determinants such as Flk-1 and Tie-2 can be utilized in the same fashion. In accordance with the present invention, any medically acceptable delivery system can be employed. Most simple and direct is the systemic introduction of the stem cells, either prior to or, preferably, after the creation of the focal injury sites. Alternatively, stem cells can be delivered to the patient via injection directly into the myocardium, into the left ventricle, intravascularly, or into the pericardial sac.

The selection and harvesting of CD 34+ cells, or similar cells is well known. Similarly, a number of different cell lines having equivalent properties to CD 34+ cells can be identified using the tools available to those of skill in the art. The introduction of a genetic construct that induces, enhances or encourages vessel growth into a CD 34+ cell, its analogue or equivalent is also well known. In its broad sense, the present invention requires binding a factor to the surface of a cell that contains an angiogenic agent, where the factor bound to the surface of the cell will result in the delivery of the cell to a site of focal injury, such as a TMR channel.

Although specific embodiments of the present invention have been specifically described, the invention is not limited to such embodiments. Upon review of the foregoing description, adaptations, modifications, variations and alternatives that utilize the spirit of the invention embodied herein will occur to those of ordinary skill. Therefore, in order to ascertain the true scope of the present invention, reference should be made to the appended claims.

What is claimed is:

1. A method of vascularizing tissue of a patient comprising:
creating one or more sites of focal injury; and
delivering hematopoietic stem cells altered to include an angiogenic agent to the patient in an amount sufficient to promote vascularization.

2. The method of claim 1, wherein the hematopoietic stem cells are selected from the group comprising Flk-1+, Tie-2+ and CD34+ cells.

3. The method of claim 1, wherein tissue is myocardial tissue, and the step of creating one or more sites of focal injury comprises delivering electromagnetic energy on the myocardium.

4. The method of claim 3, wherein the step of delivering electromagnetic energy to the myocardium comprises delivering laser energy to the myocardium.

5. The method of claim 3, wherein the step of delivering electromagnetic energy to the myocardium comprises delivering RF energy.

6. The method of claim 4, wherein the step of delivering electromagnetic energy to the myocardium comprises delivering direct current.

7. The method of claim 1, wherein the step of creating one or more sites of focal injury comprises mechanical dissection of the myocardium.

8. The method of claim 1, wherein the step of creating one or more sites of focal injury comprises delivering ultrasonic energy to the myocardium.

9. The method of claim 1, wherein the step of creating one or more sites of focal injury comprises delivering a catheter into the left ventricle of the patient.

10. The method of claim 1, wherein the step of creating one or more sites of focal injury comprises delivering a probe via an introperative procedure.

11. The method of claim 1, wherein the step of creating one or more sites of focal injury comprises delivering a probe via a minimally invasive introperative procedure.

12. The method of claim 1, wherein the step of delivering hematopoietic stem cells to the patient comprises systemic introduction of the stem cells.

13. The method of claim 1 wherein the step of delivering hematopoietic stem cells to the patient comprises injection of the stem cells directly into the myocardium.

14. The method of claim I wherein the step of delivering hematopoietic stem cells to the patient comprises injection of the stem cells into the pericardial sac.

15. The method of claim 1, wherein the step of delivering hematopoietic stem cells to the patient comprises systemic introduction of the stem cells.

16. The method of claim 1, wherein the step of delivering hematopoietic stem cells to the patient comprises introduction of cells into the left ventricle.

17. A method of amplifying the angiogenic effect of transmyocardial revascularization comprising the steps of artificially increasing the population of CD34+ mononuclear blood cells, transfecting the cells to achieve constructive expression of one or more of angiogenic cytokines or provisional matrix proteins, and introducing the cells into a patient.
